(19) United States
(12) Patent Application Publication
(10) Pub. No.: US 2024/0390149 A1
(43) Pub. Date: Nov. 28, 2024

(54) MEGA SPACER FOR SEVERE BONE LOSS AND TUMOR

(71) Applicant: Mahmoud Alm El Din HAFEZ, Giza (EG)

(72) Inventor: Mahmoud Alm El Din HAFEZ, Giza (EG)

(21) Appl. No.: 18/696,026

(22) PCT Filed: Sep. 1, 2022
(86) PCT No.: PCT/EG2022/000031

§ 371 Date: Mar. 27, 2024

(30) Foreign Application Priority Data
Sep. 28, 2021 (EG) 1539/2021

(51) Int. Cl. A61F 2/30; A61F 2/38; A61M 31/00

(52) U.S. Cl. CPC A61F 2/30724; A61F 2/3859; A61F 2/389; A61M 31/002; A61F 2002/3068; A61M 2202/04; A61M 2202/064; A61M 2205/04; A61M 2210/02

(57) ABSTRACT

A patient specific mega spacer (1, 2) for the patients having severe bone loss resulted from both of malignant and benign tumours, infection of the primary implant, or factures and severe bone loss due to trauma. The spacer was designed to fit the bone shape and anatomy based on the images captured from the CT-scan, x-ray MRI and/or ultrasound. The spacer designed with specific shape to maintain the gap and treat the infection by using a mix of antibiotics stored in reservoir (12) inside the spacer body and released with certain rate based on the medical needs. The assembly and the mechanism of the space will fit the original range of motion and recover the bone loss. The spacer manufactured by 3D printing with accredited biomaterials Nylon PA2200 to be implanted into the patient body.

### PRIOR ART

Joint replacement surgeries are one of the common surgeries that are used in cases of tumors for both malignant or benign, contamination (infection) of the primary implant, or in cases of severe fractures or severe bone loss as a result of accidents.

Joint surgeries rely on standard (conventional) instruments for performing the surgery with a limited range of sizes and shapes of the implants which finally limits the surgeon's ability to select a proper size that matches the dimensions and anatomical shape of the bone, especially in the cases of extremely small sizes and large sizes of the bones.

That doesn't guarantee the continuity of the temporary implant (spacer) successfully inside the body until it is replaced by the permanent implant.

In other scenarios, surgeons use medical cement to form an approximated shape of a spacer to fill and keep a space in the damaged bone until recovery and install the permeant implant in the human body, this scenario force the patient to stay in the bed with limited activities.

For the malignant or benign bone tumors, amputation is the last action to prevent further local spread. In cases of contamination or infection of the primary implant, and/or severe fractures or severe bone loss as a result of accidents, the situation is complicated and difficult for the patient's daily life, as he stops performing the majority of daily activities.

The limb salvage techniques have been developed to overcome this problem, as a temporary prosthesis (spacer) is implanted to match the size and volume of bone loss. However, this can only be done for adults, because for paediatric in their developmental stages cannot implant a permanent prosthesis (implant) or even implant a temporary prosthesis (spacer) for a long time inside their bodies due to their growth. This problem appears in children with childhood cancer, especially bone cancer.

The spacer implantation surgeries are pre-surgeries before the revision surgeries allowing the direct delivery of antibiotics to the infected tissues, and maintains soft tissue tension to facilitate the re-implantation procedure.

However, it faces difficulty in determining an appropriate spacer size that fit the anatomical shape of the joint or bone, and that limits the ability of the temporary prosthesis (spacer) to be inside the patient's body for a long time.

Since the optimal period for its presence inside the body is six months after the surgery. Therefore, surgeons prefer to use medical cement to make a shape that resembles and maintain the space (gap) occurred as a result of bone loss due to trauma, tumours and/or infection. From the patient perspective, the cement spacer forces the patient to stay in bed with very limited activities for a period up to three months.

From an economical point perspective, the traditional temporary prosthesis (spacer) is one of the high-priced and expensive prosthesis for the patient, as its price is close to the price of the permanent implant, which constitutes a financial burden on the patient and governments if the surgeries are performed according to the governmental health insurance regulations, which causes an increase in the number of cases on the waiting lists.

That is answer for why we need an invention like that, the invention found a method to offer and temporary prosthesis (spacer) intended for a patient instead of the bone lost as a result of severe bone loss in cases of infection, trauma and/or tumors. It is designed and manufactured based on the CT scan of the patient's knee joint and using 3D printers to produced it based on the SLS technology.

### DISCLOSURE OF INVENTION

This invention is a mega spacer for keeping the space between the damaged bone for a certain time till the recovery from the infection occurred over the primary implant, the mega spacer is also designed for patients having severe bone loss resulting from both malignant and benign tumors, or fractures and severe bone loss due to trauma.

The mega spacer is a temporary prosthesis to keep the space and allow the patient to perform his daily activities until the full recovery of infection instead of the implanting of a handmade prosthesis (spacer) which is shaped intraoperatively from medical cement by the surgeon, the cemented spacer prevents the patient from doing his daily activities as he should stay in the bed with limited movements.

The mega spacer is a patient-specific implant designed based on the anatomy of the damaged bones and the captured images coming from the X-ray, CT-Scan, MRI, and Ultrasound.

Based on the scanning images of the patient, the two-dimensional scanning images are converted and reconstructed into a three-dimensional model of the bone that is identical to the anatomical shape of the patient's bone.

The mega spacer is designed based on the volume of damaged bone to be recovered and restored, and in a parallel track, the duration of the spacer in the human body according to the infection rate and planned recovery time. the spacer is applicable to be in the human body for 6 months to 9 months without any side effects.

The mega spacer is manufactured by 3D printing and made from medical-grade material, which is selected from the Polyamide family. The manufacturing material is Nylon PA2200. The selected material has excellent mechanical and physical properties compared to many other materials, especially metal materials with a light-weight. It also has the advantage that it can be implanted inside the body and stay in it for six months to nine months, which is a sufficient and proper period for the presence of the mega spacer inside the body until the surgery of implanting the permanent prosthesis or implant.

Nylon PA2200 is a porous material that allows liquids to pass through it, this feature of the material gives the designer the opportunity to build an internal reservoir for the antibiotic to store the required amount of antibiotic needed to treat the infection.

While the manufactured material is porous, the antibiotic will be released through the body of the spacer, and through passages of releasing holes with a minuscule diameter of 0.2 mm are distributed along the spacer body. The antibiotic is released from the reservoir to the blood path and bone cavities to treat the infection. The antibiotic reservoir could fill by any type of mix of the antibiotic types.

The nylon PA2200 is internationally approved by the US Food and Drug Administration (FDA) and can be implanted inside the body without any medical complications.

The mega spacer is designed based on the CT-scan images of the patient which leads to the reconstruction of a 3D model of a bone that is identical to the patient bone, and enhances building a unique and patient-specific model which provides a perfect match with bone and strong fixation of the spacer over the bone, and perform his function of allowing the patient to practice his daily activities in an easy way.

The mega spacer consisting of two parts; femoral component (1) and tibial component (2), the two component are linked together with a hinged link (4) by inserting a rod passed through the entry hole of the hinged link rod (13) and the fixation hole of the hinged link (15) to ensure the alignment of the femoral component to the tibial component.

The hinged link (4) is blocky part seated between the formal component and tibial component to ensure the balancing and maintain the positions of the mega spacer at flexion/extension and standing/sitting positions.

The hinged link (4) has a fixation hole (15) in its body, connected to the entry hole of the hinged link rod (13) of the femoral component to the to ensure the alignment and matching along the anatomical axis of both femoral and tibial component.

The main function of the hinged link (4) is connect both the femoral component and the tibial component and restore the mechanical relation between them, as there is no connection between the femur and tibia bones of the patient due to bone loss by tumour, infection or trauma. The secondary function is to help the spacer to perform its function of keeping the space that founded due the bone loss and allowing the patient to practice his daily activities of basic movements of flexion/extension and loading on the foot.

The tibial component (2) of the mega spacer is connected to a tibial stem (3) through internal thread allowing the surgeon to assembled and disassembled intraoperatively based on the surgical needs of the patient. The tibial stem (3) has a cavity that was filled by an antibiotic in case the infection founded in the tibial shaft. The antibiotic in the tibial stem cavity released to the blood path and bone cavities through spacer body as it was made from a porous material the allowing the releasing of liquids. The antibiotic comes out spacer body with the patient's basic movements, such as walking, sitting, standing or sleeping.

The femoral component (1) has a femoral stem (17) to fix the mega spacer to the reminder part of the femur bone after the resection of damaged bone. The femoral stem (17) have a femoral medulla (5) to fix the femoral nail inside both the femur bone and the femoral component (1) of the mega spacer.

The femoral stem (17) have two alignment flanges (6) at the top of the femoral stem with two fixation holes (7) to fix the mega spacer to the femur bone by surgical transverse screws, the two alignment flanges (6) surrounded the tip of reminder part of femur bone and ensure a good fixation, which give an opportunity for a perfect seating of the femoral component (1) over the femur bone. The merit of fixation the femoral component (1) by using the two alignment flanges (6) instead of the femoral nail, is that the optimum solution in the case of high risk of using femoral nail or intra medullary rod due the sever femoral bowing and/or bleeding complications.

The femoral component is characterized by internal sound mass—the femoral component mass (14)—in order to compensate the severe bone loss. At opposite from the total knee replacement, as there an enough bone to seat the primary implant over the re-sectioned bone; here there is no volume of bone, and the surgeon needs a proper solution to recover the volume of the bone that was lost by trauma, tumour and/or infection. The femoral component mass (14) gives the surgeon the magic solution to revoke this scenario and give the mega spacer the stability to stand with the mechanical loads coming from the daily activities of the patient.

At the route of the femoral stem (17), there is an antibiotic reservoir (12), which has an internal volume of 15 cm³, which represents a quantity of approximately 8 grams of antibiotic, which is approved globally to ensure the elimination of high infection or contamination.

The antibiotic is inserted into the reservoir by medical syringe during intraoperatively, through the antibiotic injection hole (10), which is located in the femoral component (1) Postoperatively, and based on the patient's daily activities, such as walking, sitting, standing or sleeping, the antibiotics will be released from the reservoir to the blood path and bone cavities through the antibiotic release hole (11) with a minuscule diameter (0.2 mm) in order to control the rate and quantity of antibiotics released one daily based from the spacer.

In addition, the fine porous structure of the manufacturing material allows to a portion of the antibiotic to be released through the spacer body directly to the blood path and bone cavities. This feature gives the surgeon an opportunity to add or push an extra amount of the antibiotic to the blood path in the advanced cases of the tumours and sever infection over the primary implant.

The outer surface of the femoral stem (17) has protrusions (8) with 1 mm height, the rough surface of the femoral stem (17) allows the surgeon to add and coat the femoral stem surface by a paste formed by mixing medical cement with two types of the antibiotic, one is liquid based and the other is powdered based. The thicken of the coasting layer is 1 mm similar to the protrusions (8) height, which allows the surgeon to determine the volume of the antibiotic in an accurate way.

Coating the outer surface of the femoral stem (17) with antibiotic paste offering an extra antibiotic amount in cases of sever infection rates.

The femoral component (1) of the mega spacer is characterized by consisting of two side holes (9) connected through a crescent-shaped duct for inserting surgical sutures from one side to other side to fix the femoral component (1) to the femur ligaments and ensure that the mega spacer will perform the flexion/extension movements properly.

The femoral component (1) of the mega spacer is characterized by an articulating surface (19) that matches with femur bone surface anatomy which gives the surgeon the opportunity to use the femoral part only without the need to implant the tibial component in case of no damages at the tibia bone or the presence of a cancerous tumor in the femur only. In these cases, the surgeon preserves the healthy tibia bone and install to it only the femoral part of the mega spacer.

Nylon PA2200 is a radiolucent material and doesn't appear under the X-ray, which means that the details of the mega spacer are not visible under the x-rays. The surgeon needs to follow-up the patient postoperatively and apply some imagine process to monitoring the performance e of the mega spacer. An extra surgical wire will be inserted into the suture (surgical wire) entry holes (18) to overcome this problem and allowing the surgeon to monitoring the position and angulation e of the mega spacer in easy and accurate way under x-ray examination.

The mega spacer manufactured by 3D printer by using the Selective Laser Sintering SLS technology, while the products are printed by sintering nylon powder by using the laser beams.

### DRAWING DESCRIPTION

FIG. 1: 2D side view of the mega spacer shows the main component which are the femoral component (1), the tibial component (2), the tibial stem (3) and the femoral stem (17).

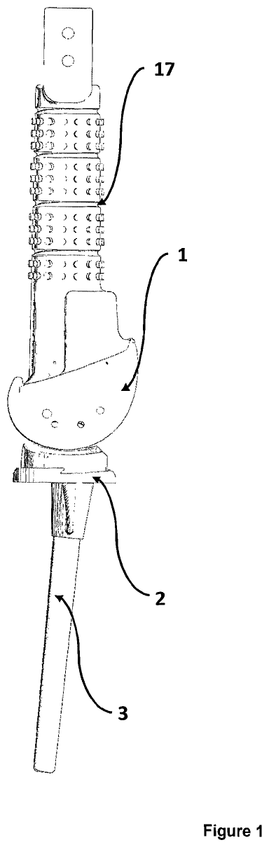

FIG. 2: 2D sectional view of the mega spacer shows the femoral component (1), the tibial component (2), the tibial stem (3), the alignment flanges of the femoral component (6), the fixation hole of the femoral component (7), femoral medulla (5), hinged link (4), antibiotic reservoir (12), the tibial stem cavity (16), the femoral stem (17) and the suture (surgical wire) entry holes (18).

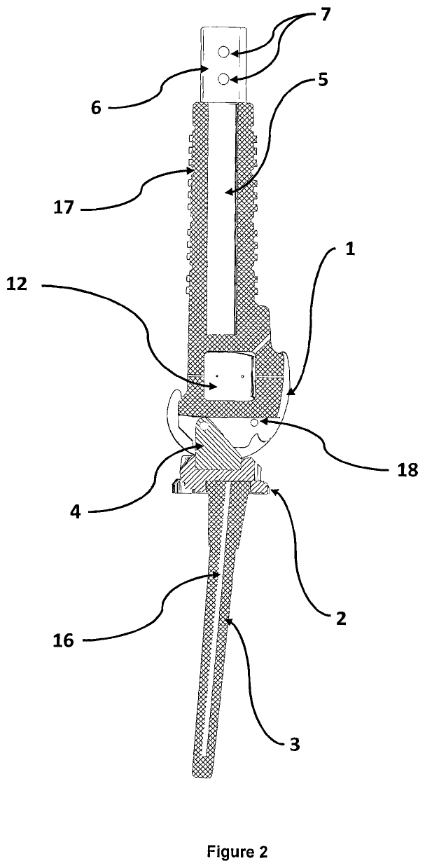

FIG. 3: 3D isometric view of the mega spacer shows the femoral component (1), the tibial component (2), the tibial stem (3), the femoral component protrusions (8), Hinged link (4), and the femoral stem (17).

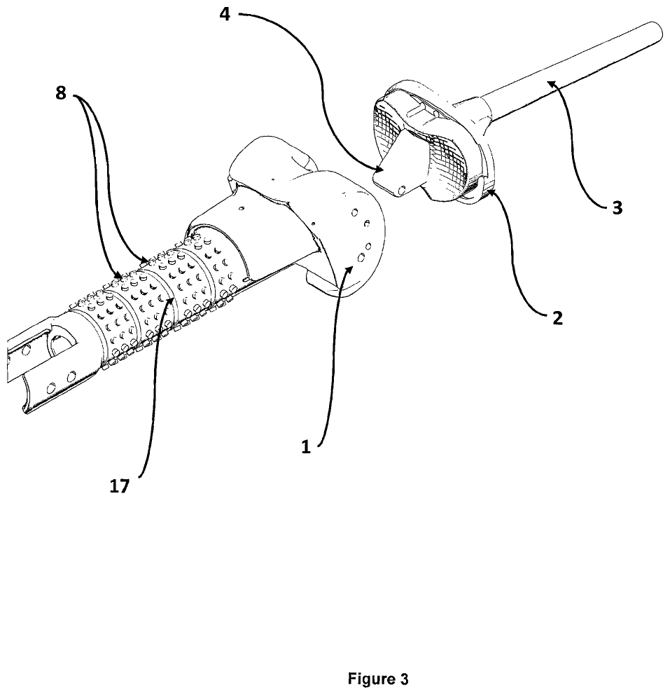

FIG. 4: 3D isometric view for femoral component of the mega spacer shows the femoral component (1), femoral medulla (5), femoral component (6), the fixation hole of the femoral component (7), the femoral component protrusions (8), the suture (surgical wire) entry holes (18), antibiotic injection hole (10), antibiotic release holes (11), femoral component mass (14), fixations holes of femoral component to ligaments (9), and entry hole of the hinged link rod (13).

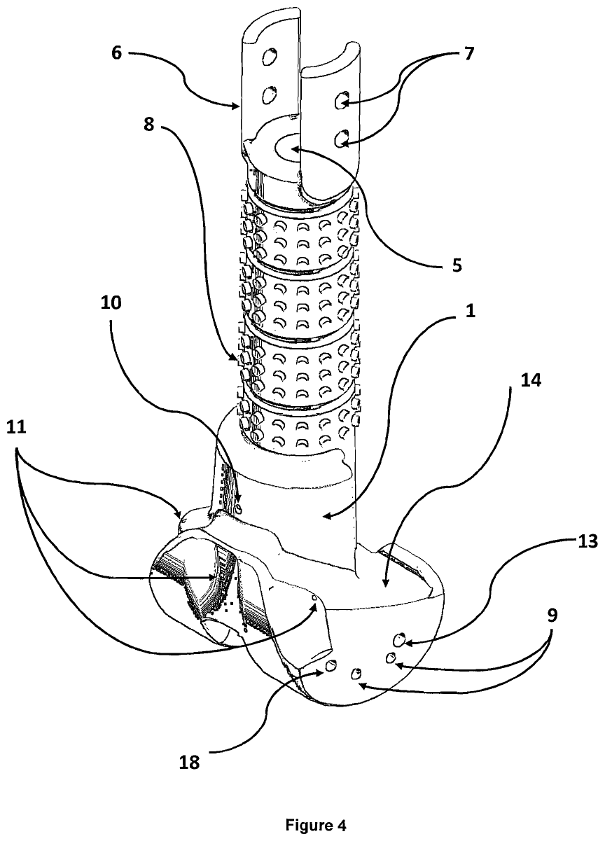

FIG. 5: 2D side view for femoral component of the mega spacer shows the antibiotic injection hole (10), antibiotic release holes (11), antibiotic reservoir (12), the suture (surgical wire) entry holes (18), entry hole of the hinged link rod (13) and articulating surface of the femoral component (19).

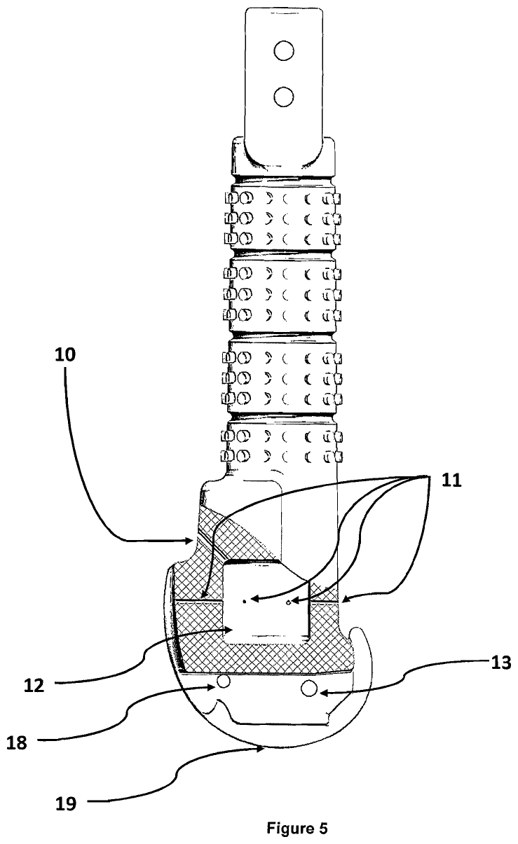

FIG. 6: 2D sectional view for femoral component of the mega spacer shows fixations holes of femoral component to ligaments (9).

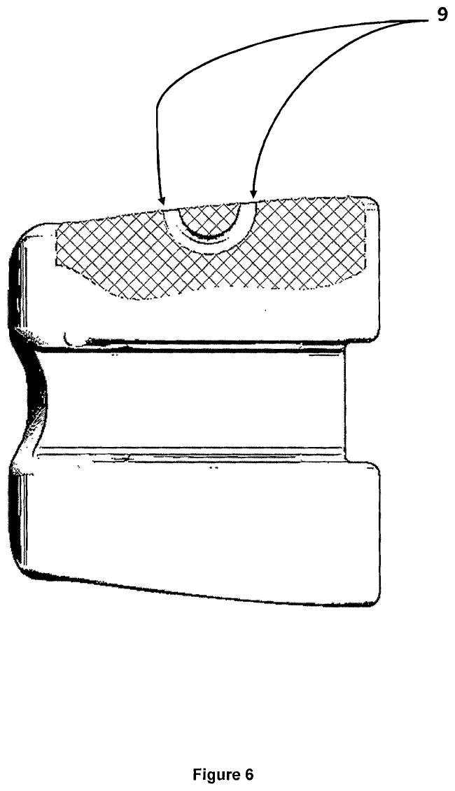

FIG. 7: 3D isometric view of the mega spacer in the flexion position shows the femoral component (1), the tibial component (2), the tibial stem (3), hinged link (4), and entry hole of the hinged link rod (13).

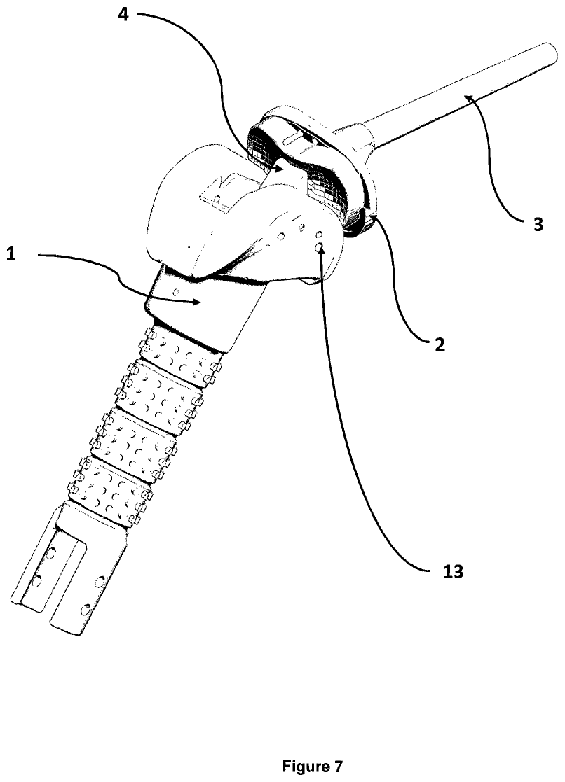

FIG. 8: 3D isometric view of hinged link (4) shows the fixation hole of the hinged link to the femoral component of the mega spacer (15).

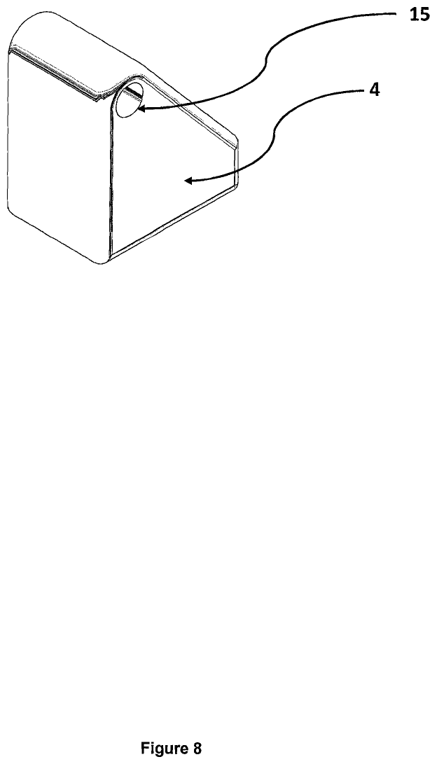

1- A spacer for a patient having severe bone loss resulting from malignant and benign tumours, infection of the primary implant, or fractures and severe bone loss due to trauma comprising:
a femoral component (1) and a tibial component (2), the femoral component including an antibiotic reservoir (12), surface protrusions on a femoral stem (8), surgical wire entry holes (18), and antibiotic injection and releasing holes (10 and 11 respectively).

2- The spacer according to claim 1, wherein the femoral component (1) and tibial component (2) are linked together by a hinged link (4) formed by a rod passed through an entry hole of a hinged link rod (13) and a fixation hole of the hinged link (15) to ensure alignment of the femoral component to the tibial component.

3- The spacer according to claim 1, wherein the femoral component (1) of the spacer is characterized by the antibiotic reservoir (12) adapted to be filled with an antibiotic by a medical syringe injecting a planned dose through an antibiotic injection hole (10).

4- The spacer according to claim 1, the femoral component (1) of the spacer is characterized by antibiotic release holes (11) with a diameter of 0.2 mm to ensure a fixed rate of antibiotic release.

5- The spacer according to claim 1, wherein the spacer is made from a porous material to allow an antibiotic to be released from the antibiotic reservoir to the bone cavities.

6- The spacer according to claim 1, wherein the femoral component (1) of the spacer is characterized by surface protrusions (8) on the femoral stem with a height of 1 mm which surface protrusions force the paste to stick to a surface of the femoral stem.

7- The spacer according to claim 1, wherein the femoral component (1) of the spacer is characterized by a femoral medulla (5) of the femoral stem to fix a femoral nail inside both a femur bone and the femoral component (1) of the spacer.

8- The spacer according to claim 1, wherein the femoral component (1) of the spacer is characterized by two alignment flanges (6) at a top of the femoral stem with two fixation holes (7) to fix the spacer to a femur bone with transverse screws.

9- The spacer according to claim 1, wherein the femoral component (1) of the spacer is characterized by two side holes (9) connected through a crescent shaped duct for inserting surgical sutures to fix the femoral component (1) to femur ligaments.

10- The spacer according to claim 1, wherein the femoral component (1) of the spacer is characterized by an articulating surface (19) that matches with a femur bone surface anatomy which allows a surgeon to implant the femoral component without the need to implant the tibial component in case of no damage at a tibia bone.

11- The spacer according to claim 1, wherein the tibial component (2) of the spacer is connected to a tibial stem (3) by an internal thread that can assembled and disassembled according to the need intraoperatively, the tibial stem (3) having a cavity (16) filled with an antibiotic, while the antibiotic release through a porous material of the tibial stem (3) is based on the patient's daily movements of flexion, extension, standing and sitting.

12- The spacer according to claim 1, further including an antibiotic paste coating comprising two different types of antibiotics mixed together with medical cement, one of the antibiotics being a powdered based and the other being a liquid based.
